(19) United States
(12) Patent Application Publication
(10) Pub. No.: US 2007/0196831 A1
(43) Pub. Date: Aug. 23, 2007

(54) Human sample matching system

(76) Inventors: Edward F. Tuck, West Covina, CA (US); Martie G. Haselton, Culver City, CA (US)

Correspondence Address: Giaccherini, Post Office Box 1146, Carmel Valley, CA, 93924, US

(21) Appl. No.: 11/360,025

(22) Filed: Feb. 21, 2006

(51) Int. Cl. C12Q 1/68; G06F 19/00

(52) U.S. Cl. 435/6; 702/20

(57) ABSTRACT

Methods and apparatus for a human sample (S), analyzing the sample (S) and then determining a match with a member of the opposite sex is disclosed. In one embodiment, a customer purchases an AromaMatch™ Test Kit (14), which comprises a bottle of cleaning solution (20), a cotton ball (22) a sample patch (24), a sample bag (28) and a mailing envelope (30). The user (10) opens the kit (14), cleans a patch of skin somewhere on his or her body, and then applies the patch (24). After wearing the patch (24) for an appropriate time period, the patch (24) is removed, and placed in the sample bag (28). The user (10) writes his or her password on the sample bag (28), and then mails it in the return envelope (30). A laboratory analyzes the sample patch (24), and determines a set of genetic attributes (G1) that are associated with the sample (S). The user (10) receives an analysis report by mail, or views the results of the analysis on a website (18), such as www.AromaMatch.com. In another embodiment, a customized perfume is manufactured that is based on biological agents that are selected to match the set of genetic attributes identified by the analysis.

### FIELD OF THE INVENTION

The present invention pertains to methods and apparatus for using a human sample to predict a good match between a man and a woman. The human sample may comprise an odor or an actual tissue or cell sample. More particularly, the invention takes advantage of the human female's enhanced sexual response to men who possess a particular genetic profile that differs from her own, and the heightened attractiveness of either sex to such differing profiles. Specifically, a romantic match between a man and a woman is predicated on a strong correlation among a specific cluster of human genes called the “Major Histocompatibility Complex (MHC).”

### BACKGROUND OF THE INVENTION

Mammals have evolved efficient ways to find and select among potential mates. There has been a great deal of research on this subject in the twenty-three years since a landmark study found that mice choose their mates on the basis of their candidates' distinctive odors. Boyse E A, Beauchamp G K, Yamazaki K, et al., “Chemosensory Communication—A New Aspect of the Major Histocompatibility Complex and Other Genes in the Mouse.” Journal: Oncodevelopmental Biology and Medicine. Vol. 4 No. 1-2: pages 101-116, 1982. These odors are defined by the Major Histocompatibility Complex (MHC). The MHC is a cluster of genes that determines details of cellular surfaces and thus immune responses, and specifies certain peptides that appear in skin secretions and urine. These peptides are responsible for odors which uniquely identify individuals who are not identical twins.

More recent work has shown that human female sexual responsivity to a male partner varies linearly and inversely with the degree to which genes in the Major Histocompatibility Complex are shared. Garver-Apgar, Christine E. et al., “MHC Alleles, Sexual Responsivity, and Unfaithfulness in Romantic Couples,” Psychological Science. (manuscript in press). The correspondence is dramatic: about a nine-to-one difference in responsivity to men who share none of her MHC genes and to those who share sixty percent.

Men and women detect others' MHC genes through their body odors. There are a number of peptides that are derived from particular regions of the MHC. These peptides are detected as odors. They strongly affect a woman's responsivity to a particular partner, as discussed in the cited literature, and to both men's and women's mutual attractiveness.

This mate-selection process has a strong effect on the fitness of offspring. Choosing mates on the basis of MHC dissimilarity equips offspring with a broad immune system, increasing the offspring's fitness. It also selects against close relatives as mates, increasing the viability of fetuses and reducing birth defects. It also may reduce the rate of spontaneous abortion: there is some evidence that fetuses of couples which share significant numbers of MHC alleles are more likely to be rejected in utero. Zeh, Jeanne A. et al., “Outbred embryos rescue inbred half-siblings in mixed-paternity broods of live-bearing females,” Nature, Vol. 439, pp. 201-203 (12 Jan. 2006).

Other studies, including one cited above, have shown that women who are in long-term intimate relationships with men with similar MHCs are more likely to seek other partners during the fertile portion of their menstrual cycles. This practice obviously has a destabilizing effect on these relationships, which include marriages. Because humans' sense of smell is relatively poor, couples who are strangers must come into close personal contact before he or she can estimate their MHC-derived “fit” with a potential male partner and thus her long-term sexual responsivity to him. As humans have moved from villages to cities, various means have been created to bring men and women of marriageable age into close proximity under controlled conditions: examples range from the masked ball in Romeo and Juliet to modern on-line dating services. In modern human society, with much less class structure and much more freedom for men and women than in primitive, medieval or Victorian eras, this acquaintance process can pose considerable danger and risk of embarrassment to women. The modern process of selecting a mate is very inefficient compared to these earlier societies, in which the number of potential partners available to each woman was comparatively small and where people lived in very close proximity. It would be of great benefit, not only to individual couples, but to society as a whole, if men and women could assess the sexual compatibility of prospective mates without coming into close contact. This would, among other things, give women a wider range of prequalified candidates and would give men greater assurance that they and their prospective mates would have a stable and persistent relationship characterized by mutual physical attraction. It is generally conceded that mutual sexual attraction and responsivity are major contributors to pair bonding: they are the glue that holds long-term relationships together. People of all political and religious persuasions agree that stable pair-bonding, carrying the benefit of reduced strife and relationship discord, is in the best interest of society. Strife and relationship discord result in failed marriages and in infidelity. Society as a whole will thus benefit from easier and more accurate receptivity assessment. It is also important to note that there remain many cultures in which arranged marriages are the norm, and affianced couples do not meet before their wedding ceremony. Parents and matchmakers who are concerned with the success of their efforts could gain confidence from an MHC-based genetic matching process before a commitment is made.

Technology has advanced to the point that individual MHC-derived peptides, and thus odors, can be accurately detected artificially using gas chromatography and/or mass spectrometry (an “e-nose”). Willse, Alan et al., “Identification of Major Histocompatibility Complex-Regulated Body Odorants by Statistical Analysis of a Comparative Gas Chromatography/Mass Spectrometry Experiment,” Analytical Chemistry, Vol. 77, No. 8 (Apr. 15, 2005). This implies that a personal odor profile can be constructed for each individual, and that the degree of MHC sharing of two individuals can be derived by comparing those measurements, even if they are strangers and geographically distant from one another. MHC analysis can also be done on the basis of other material, such as cheek-cell scrapings, saliva tests, and other means used in forensic settings.

This process represents a considerable improvement to acquaintance-facilitation (“dating”) services based on the use of questionnaires and personality profiling. While these services help people find partners based on their subjective preferences and personality match, they say little about the likelihood of sexual attraction on first meeting, or the sexual responsivity of the partners in a long-term relationship. In contrast to these methods, MHC comparison is a completely objective process. Unlike current processes which rely on self-administered questionnaires, remote psychological assessments and other user-supplied personal data, MLC comparison cannot misrepresent its user.

The development of a system that could take advantage of this biological predilection of women to exhibit an enhanced attraction and sexual response to men who share her own genetic attributes would fulfill a long felt need in the dating and relationship industry, and would constitute a great benefit to members of society.

### SUMMARY OF THE INVENTION

The present invention provides a method and apparatus for taking a human sample, analyzing the sample and then determining a match with a member of the opposite sex. In one embodiment, a customer purchases an AromaMatch™ Test Kit, which comprises a bottle of cleaning solution, a sample patch, a sample bag and a mailing envelope. The user opens the kit, cleans a patch of skin somewhere on his or her body, and then applies the patch. After wearing the patch for an appropriate time period, the patch is removed, and placed in the sample bag. The user writes his or her password on the sample bag, and then mails it to a laboratory in the return envelope. The laboratory analyzes the sample patch, and determines a set of genetic attributes that are associated with the sample. The user receives an analysis report by mail, or views the results of the analysis on a website, such as www.AromaMatch.com.

In an alternative embodiment of the invention, the tissue sample may be obtaining using a cheek swab. Other methods of obtaining a sample, including the use of blood, urine, hair or saliva, may be employed to implement the present invention.

In another alternative embodiment, a kiosk or collector may be used to collect an aroma sample directly from the air, or from a cell sample collected locally.

In yet another embodiment, the customer may purchase a custom-formulated perfume, cologne, salve or other cosmetic or preparation that contains enhanced aromas that match his or her genetic attributes.

An appreciation of the other aims and objectives of the present invention, and a more complete and comprehensive understanding of this invention, may be obtained by studying the following description of preferred and alternative embodiments, and by referring to the accompanying drawings.

### A BRIEF DESCRIPTION OF THE DRAWINGS

FIG. 1 shows a woman purchasing an AromaMatch™ Test Kit at a retail store.

FIG. 2 shows the same woman purchasing an AromaMatch™ Test Kit on a website.

FIG. 3 depicts the woman opening the AromaMatch™ Test Kit to reveal its contents: a bottle of cleaning solution, a cotton ball, a sample patch, a sealable plastic bag and a mailing envelope.

FIG. 4 supplies a detailed view of the sample patch.

In FIG. 5, the woman cleans a patch of skin in preparation for applying the sample patch to her arm.

In FIG. 6, the woman applies the patch to her forearm.

FIG. 7 shows the woman wearing the patch all day.

FIG. 8 portrays the woman removing the sample patch from her arm later that evening.

In FIG. 9, the woman places the sample patch that she has worn for a day into the bag, and seals it.

In FIG. 10, the woman writes her password on the sealable bag.

In FIG. 11, the sample that has been sealed in the bag is placed in a mailing envelope.

FIG. 12 shows the woman mailing an envelope which contains the bag, which, in turn, contains the worn sample patch.

FIG. 13 shows a laboratory technician using an analyzer to determine the genetic attributes of the tissue sample that has been received from the woman depicted in FIG. 12.

In FIG. 14, the woman uses her computer to visit a website to obtain the results of the laboratory analysis.

In FIG. 15, the website reports the results of a matching process that has been performed using a library of candidates.

FIG. 16 exhibits an alternative embodiment, which collects a sample directly from the air surrounding a man.

FIG. 17 reveals yet another alternative embodiment, in which a tissue sample is obtained using a cheek swab.

In FIG. 18, a woman whose tissue sample has already been analyzed receives a custom-formulated perfume which contains aromas that correspond to her genetic attributes.

FIG. 19 shows a man using a MateFinder™ device which has been programmed with his genetic attributes, as determined in accordance with the present invention.

FIG. 20 shows a woman visiting a doctor's office or an independent laboratory to provide a tissue sample, which is then certified by the doctor or laboratory technician before it is submitted for analytic comparison.

FIG. 21 depicts a method of manufacturing a customized perfume.

### A DETAILED DESCRIPTION OF PREFERRED & ALTERNATIVE EMBODIMENTS

I. The AromaMatch™ System

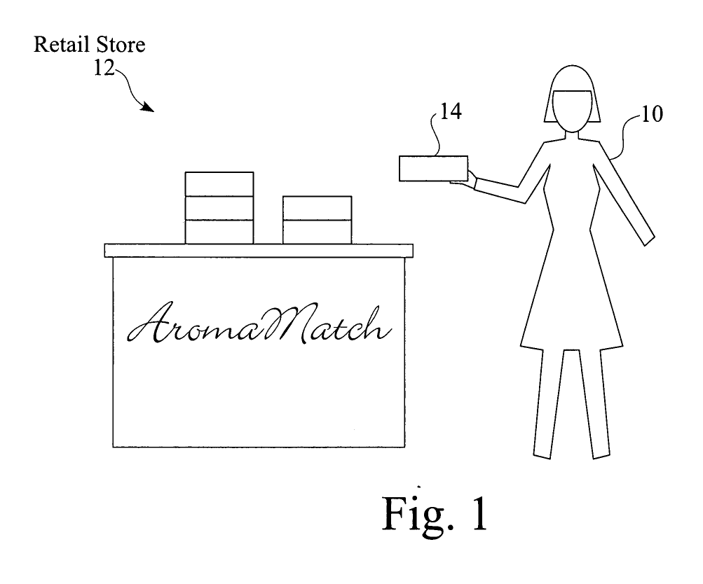

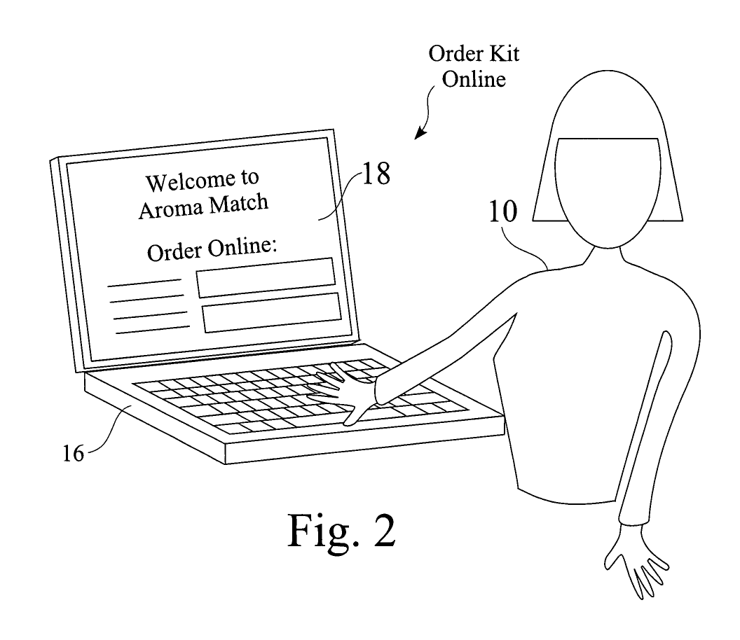

FIG. 1 offers a view of a woman 10 in a retail store 12, where she purchases an AromaMatch™ Test Kit 14. As an alternative, the woman 10 may purchase the same AromaMatch™ Test Kit 14 by visiting a website, www.AromaMatch.com, using her personal computer, as shown in FIG. 2. “AromaMatch” is a Trade & Service Mark owned by the Assignee of the Present Patent Application. The website “www.aromamatch.com” is also owned by the Assignee of the Present Patent Application. In this Specification and in the Claims that follow, the term “aroma” encompasses any scent, smell, odor or olfactory component that may or may not be actively or consciously detected, sensed or smelled by a person.

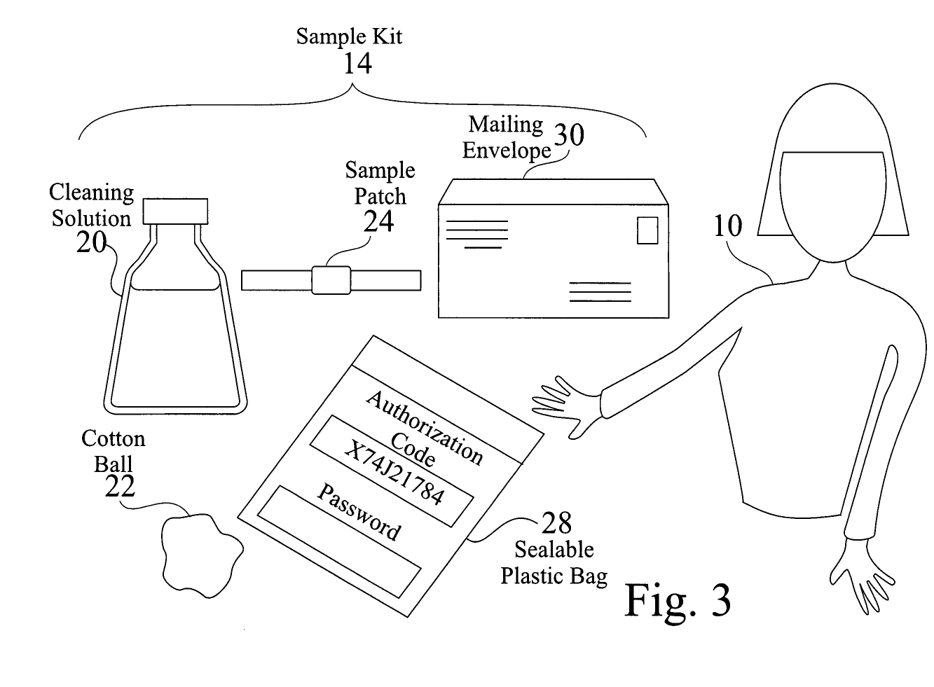

The woman opens the Test Kit 14 and removes the contents in FIG. 3. In one embodiment of the invention, the Test Kit 14 comprises:

skin cleaner 20;

a cleaning medium 22;

a sample patch 24;

a sealable enclosure 28; and

a mailing envelope 30.

The skin cleaner 20 may comprise a liquid cleaning solution such as isopropyl alcohol, or any other, gel, solid, spray or substance that cleans and/or sterilizes a portion of the skin. The application of the skin cleaner 20 removes or neutralizes perfumes and other irrelevant smells.

The cleaning medium 22 is generally a small portion of material that is used to apply the skin cleaner 20 to the skin. In one embodiment, the cleaning medium 22 may be a cotton ball, wad, paper, piece of fabric or some other suitable application device.

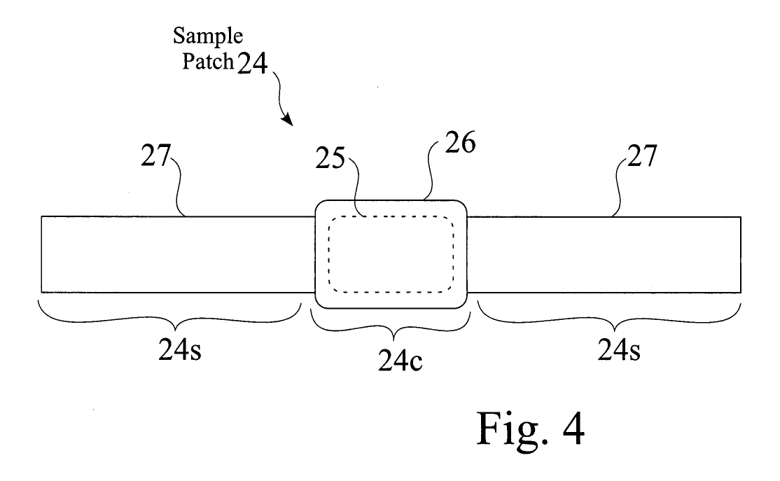

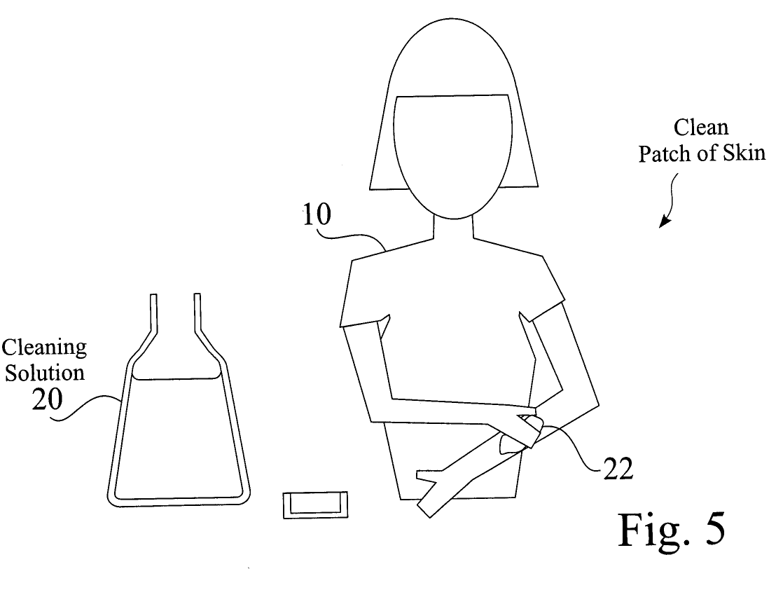

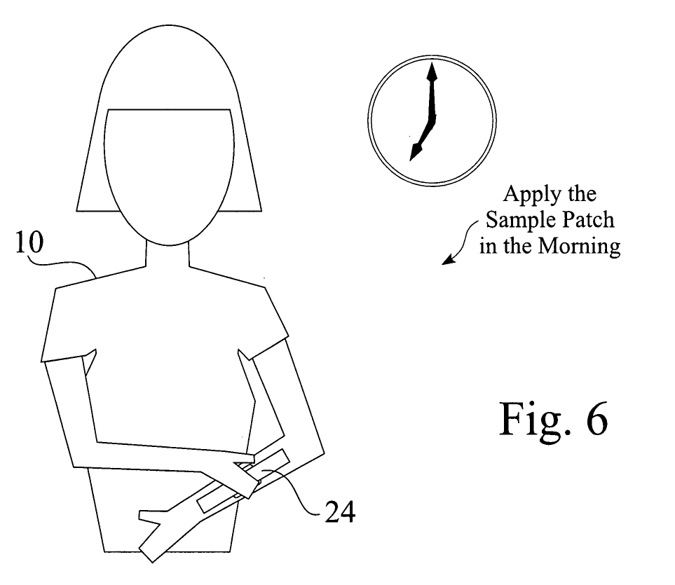

FIG. 4 furnishes an illustration of the sample patch 24, which comprises a small central area 24C with two outwardly extending strips 24S. The central area 24C is coated with a portion of plaster 25 which, in turn, has been coated with an antibiotic 26 or some other suitable agent that prevents bacterial growth which might modify the aroma. The strips 24S on either side of the plaster 25 are coated with an adhesive 27 that is suitable for adhering to the skin for a short period of time.

In one embodiment, the patch 24 resembles a conventional “Band-Aid® Brand” Adhesive Bandage, such as that manufactured and sold by Johnson & Johnson of New Brunswick, N.J. The patch 24 may be fabricated from plastic, cloth, paper or any other material that will maintain the plaster 25 in generally continuous contact with the skin. The plaster 25 is generally any material that will absorb and then hold an aroma which has been secreted by the skin. The plaster 25 may be composed of any substance that collects and stores an aroma. In one embodiment of the invention, the plaster 25 is manufactured from any material that may be used as an odor-absorbing poultice.

The plaster 25 is designed so that it will collect enough aromas to provide a sample which may be reliably analyzed. The aromas captured by the plaster 25 must be able to survive for a duration of time that is required for the patch 24 to be mailed to a laboratory.

After the Test Kit 14 is opened, the woman 10 cleans a patch of skin on her arm in preparation for applying the sample patch 24, as shown in FIG. 5. In FIG. 6, patch 24 has been attached to her forearm. The patch 24 may be worn on any portion of the body which allows direct and intimate contact with the skin. In some instances, selecting a patch of skin on or near the armpits may be produce optimal results.

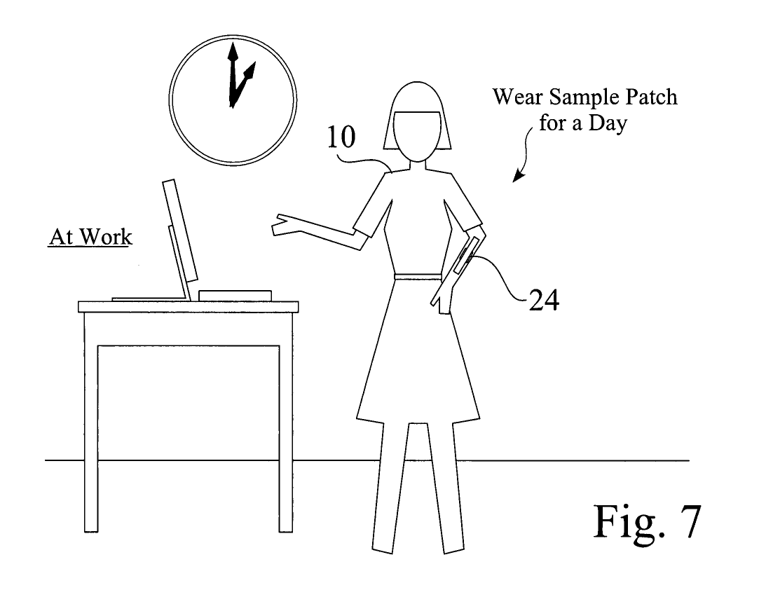

The woman wears the patch 24 all day, as shown in FIG. 7. The time that is required for the patch 24 to remain in place varies with the effectiveness of the plaster 25 and the sensitivity of the equipment used to analyze the patch 24. In one embodiment of the invention, the user is instructed to leave the patch 24 in place on the skin for at least eight hours. In some instances, the time that is required to wear to patch to obtain a good sample may take longer. One alternative method that may be used to collect a sample is using a simply wearing a shirt or some other article of clothing for an extended time, and then analyzing this worn article of clothing.

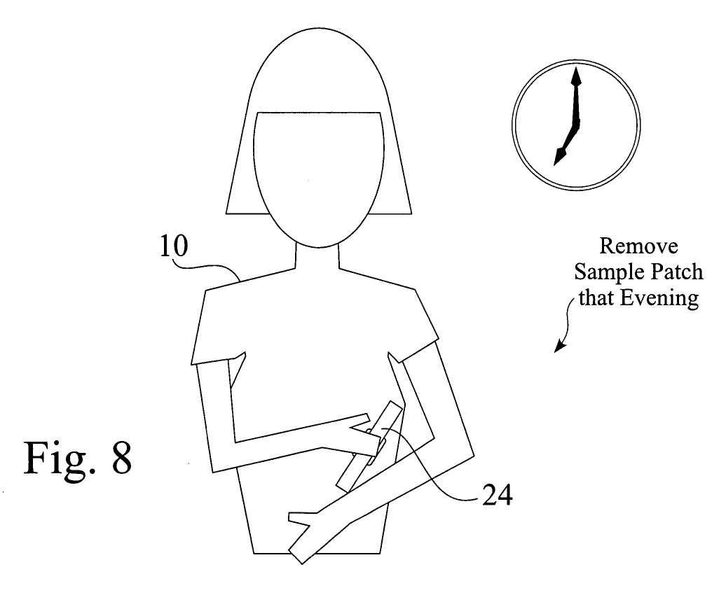

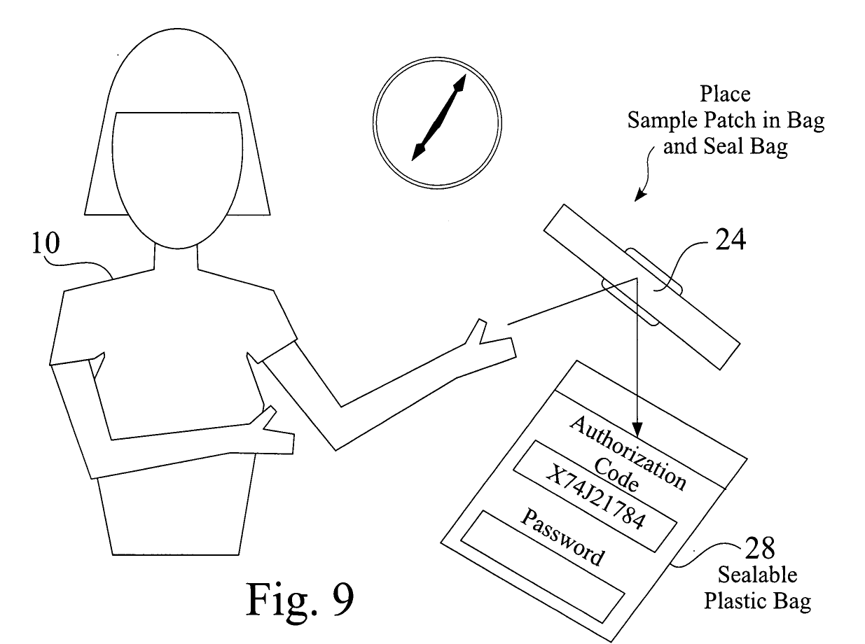

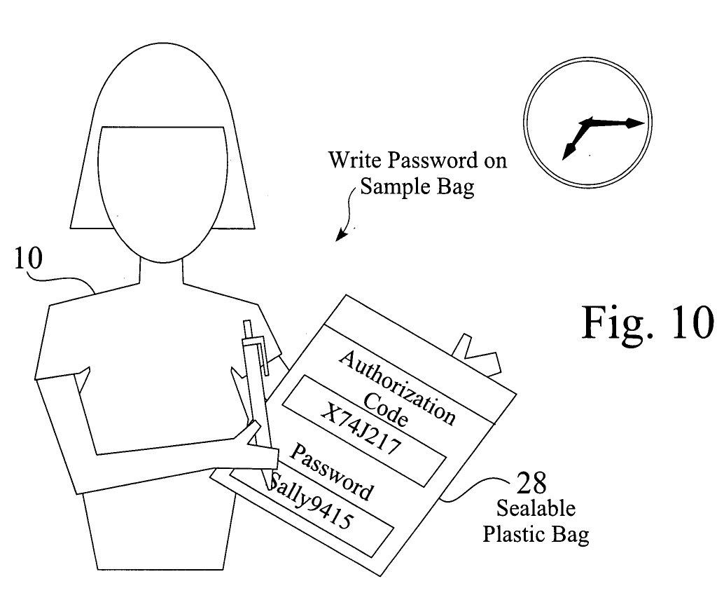

After wearing the patch 24 all day, the woman 10 removes the patch 24 later that evening, as shown in FIG. 8. After the patch 24 is removed, she then immediately places the patch 24 in the enclosure 28, as illustrated in FIG. 9. The enclosure is sealed 28 to prevent any degradation of the aromas stored in the plaster 25.

She then writes her username, password, code or some other identifying information on the bag 28, as shown in FIG. 10. This enclosure 28 is large enough to hold the sample patch 24, may be easily sealed against the intrusion of outside air by the user, and is generally an impermeable container or barrier that preserves the aromas imparted to the plaster 25 on the patch 24. In one embodiment of the invention, the enclosure 28 is a plastic bag with a compression seal, which is commonly known as a “zip-lock” or “slide-lock” closure. In one implementation, the bag 28 bears a pre-printed authorization code.

The patch 24 which stores the sample S which has been sealed in the bag 28 is then placed in the mailing envelope, as shown in FIG. 11.

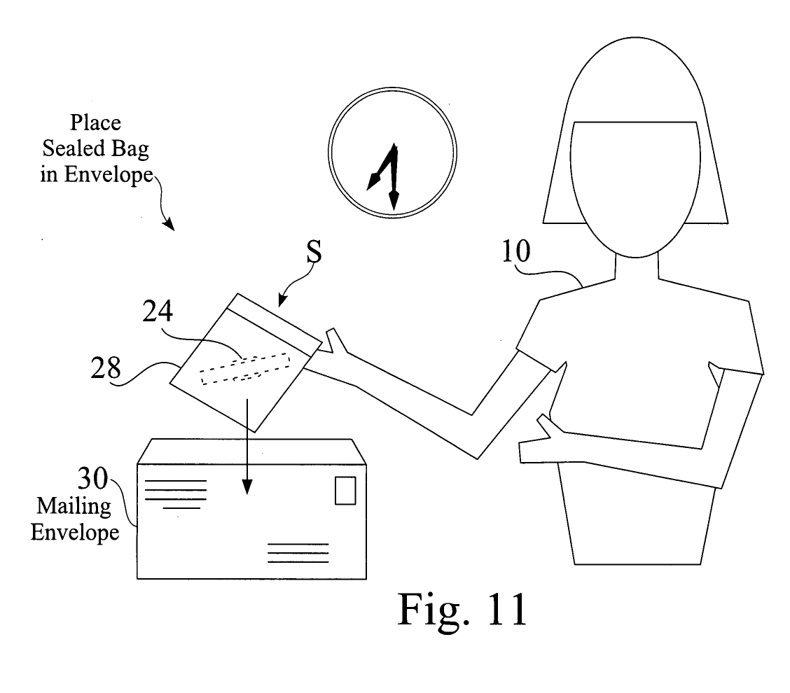

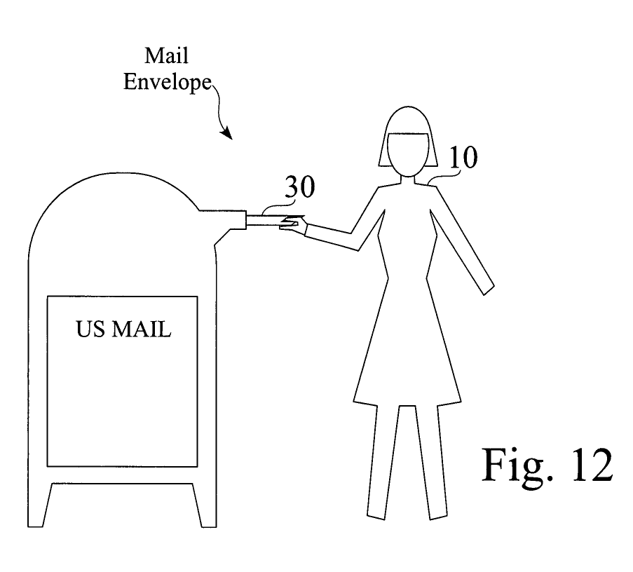

FIG. 12 portrays the customer posting the pre-addressed mailing envelope 30 which contains the worn patch 24 in the bag 28. This envelope 30 will convey the patch 24 to a laboratory where the plaster 25 will be analyzed. As an alternative, the patch 24 may be shipped to a laboratory using a courier. The patch 24 may also be delivered to a local lab, doctor's office or pharmacy for analysis. In a more advanced embodiment of the invention, the user may analyze the patch 24 using a home analysis kit.

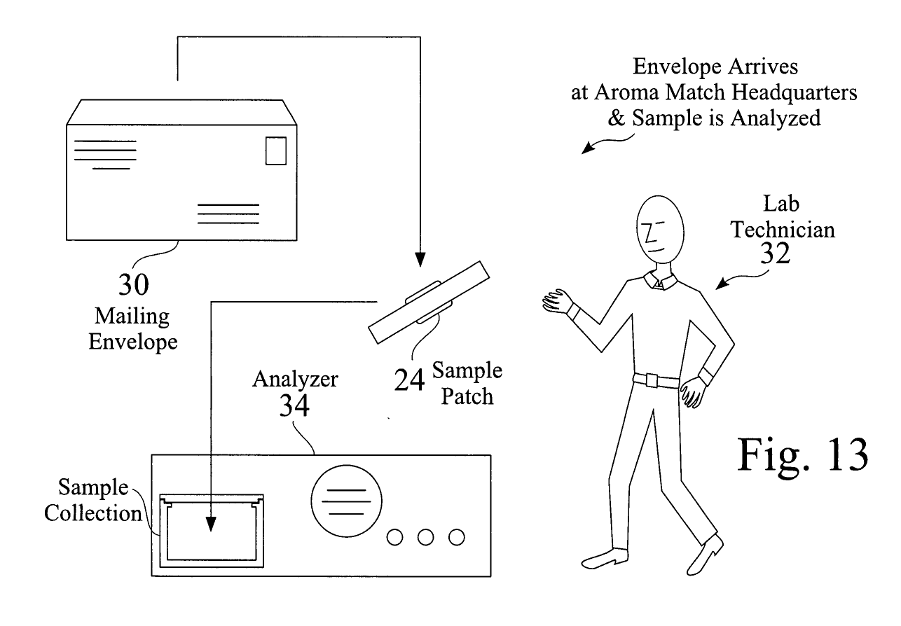

FIG. 13 shows a laboratory technician 32 using an analyzer 34 to determine the genetic attributes of the tissue sample that has been received from the customer 10. In one embodiment, a probe from an analyzer 34 may be inserted into the bag 28, which will convey the aromas to a chamber where a chemical analysis is conducted.

Several devices and systems for analyzing a sample are currently available which may be used to implement the present invention. One device called an “Electronic Nose” has been described by The Lewis Group of The California Institute of Technology, and is based on readily fabricated, chemically sensitive conducting polymer films. According to information presented on their website:
“An array of sensors that individually respond to vapors can produce a distinguishable response pattern for each separate type of analyte or mixture. Pattern recognition algorithms and or neural network hardware are used on the output signals arising from the electronic nose to classify, identify, and where necessary quantify, the vapor or odors of concern. This response is much like the way the mammalian olfactory sense produces diagnostic patterns and then transmits them to the brain for processing and analysis.
This approach does not require development of highly specific recognition chemistries, one for each of the many possible analytes of interest. Instead this approach requires a broadly responsive array of sensors that is trainable to the target signature of interest and then can recognize this signature and deliver it to the sensing electronics in a robust fashion for subsequent processing by pattern recognition algorithms. The Caltech electronic nose functions at atmospheric pressure, functions in a variety of ambients, exhibits near-real time detection, and has already been demonstrated to track vapors in air.
The underlying principle of the Caltech electronic nose is extraordinarily simple. When a polymer film is exposed to a gaseous vapor, some of the vapor partitions into the film and causes the film to swell. In the electronic nose, this swelling is probed electrically because the sensor films each consist of a composite that contains regions of a conductor that have been dispersed into the swellable organic insulator. The vapor-induced film swelling produces an increase in the electrical resistance of the film because the swelling decreases the number of connected pathways of the conducting component of the composite material. The detector films can be formed from conducting polymer composites, in which the electronically conductive phase is a conducting organic polymer and the insulating phase is an organic polymer, or from polymer-conductor composites in which the conductive phase is an inorganic conductor such as carbon black, Au, Ag, etc and the insulating phase is a swellable organic material. The electrical resistance of the device is then read using simple, low power electronics.
Any individual sensor film responds to a variety of vapors, because numerous chemicals will partition into the polymer and cause it to swell to varying degrees. However, an array of sensors, containing different polymers, yields a distinct fingerprint for each odor because the swelling properties over the entire array are different for different vapors. The pattern of resistance changes on the array is diagnostic of the vapor, while the amplitude of the patterns indicates the concentration of the vapor.”
   See: The Lewis Group, California Institute of Technology, Pasadena, Calif. Webpage: http://nsl.caltech.edu/resnose.html

A second device that may be used to implement the present invention is called the “Cyranose,” and is described by Rodney M. Goodman, in his article entitled “The Electronic Nose.” According to Goodman:
“Cyranose
The technology uses sensors mixed with carbon black to make them conductive. The polymers swell with an odorant and their resistance changes. An array of different polymers swell to different degrees giving a signature of the odorant. This technology has been commercialized by Cyrano Sciences (http://cyranosciences.com) and a handheld electronic nose has been launched as a product.”
   See: http://www.rodgoodman.ws/electronic_nose.html

A third device that may be used to implement the present invention is described by Smiths Detection of Danbury, Conn., which produces and sells devices for identifying materials. See: http://www.sensir.com/Smiths/Home.htm.

In FIG. 14, the customer 10 uses her computer 16 to visit a website 18 to obtain the results of the laboratory analysis. In one embodiment, the analysis includes a listing of MHC alleles, MHC-determined peptides, MHC-odors or some other MHC-dependent profile. In an alternative embodiment, the results may be dispatched to the customer by regular mail or by e-mail.

In an alternative embodiment of the invention, the customer pays for the Test Kit 14 and the analysis when he or she obtains the results of the analysis.

In FIG. 15, the website 18 reports the results of a matching process that has been performed using a library of candidates.

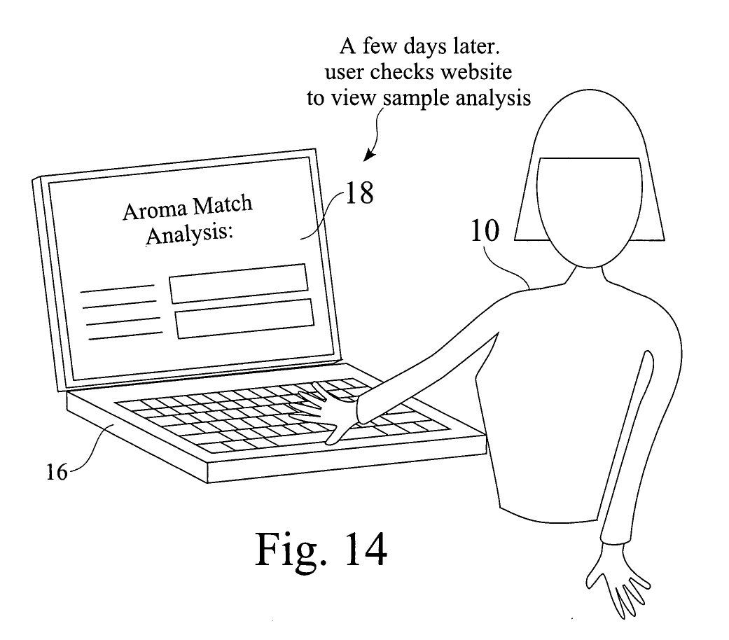

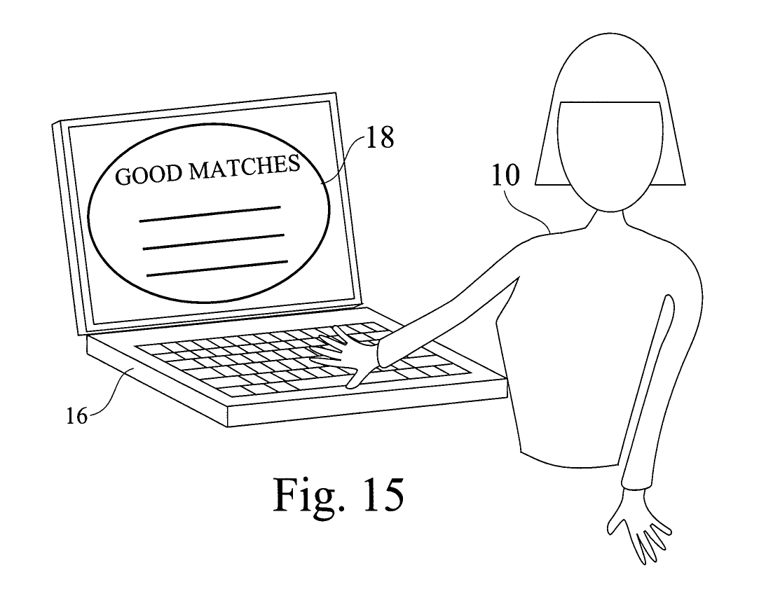

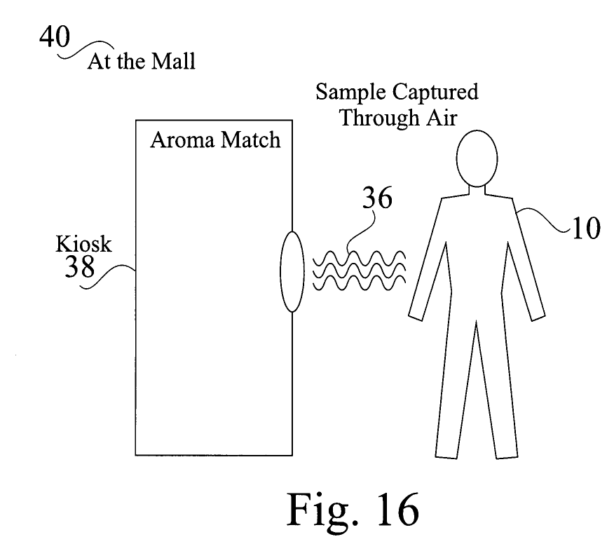

FIG. 16 exhibits an alternative embodiment, which collects a sample directly from the air 36 surrounding a customer 10 standing near a kiosk 38 that has been installed in a shopping mall 40. In yet another embodiment, a sample collecting tube may briefly be placed under a portion of a customer's clothing to obtain an air sample.

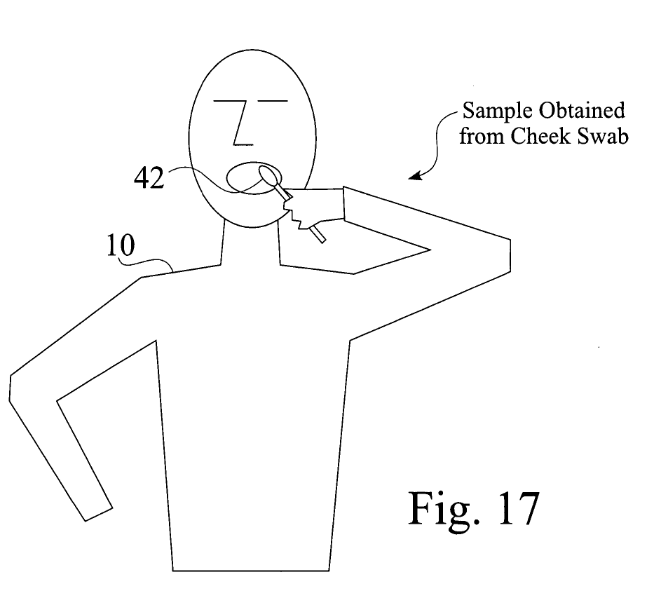

FIG. 17 reveals yet another alternative embodiment, in which a tissue sample 42 is obtained using a cheek swab. In other embodiments, a tissue sample may be obtained from any suitable bodily material or fluid, including, but not limited to, blood, saliva, exhaled breath, fingerprint, urine, hair, nail, or skin. One device that may be used to implement this portion of the present invention is produced and sold by DNA Genotek of Ottowa, Ontario, Canada, which produces and sells the Oragene™ DNA Self-Collection Kit, for collecting and preserving large amounts of DNA from saliva. See: http://www.dnagenotek.com/

In an alternative embodiment of the invention, an automatic machine or device which accepts a DNA sample may be used to obtain an analysis without the intervention of a technician or clerk.

In FIG. 18, a customer whose tissue sample has already been analyzed receives a custom-formulated perfume 44, “MyAroma™” or “MyCologne™,” which contains olfactory reagents that correspond to her genetic attributes, and specifically, which correspond to his or her MHC-derived peptide profile.

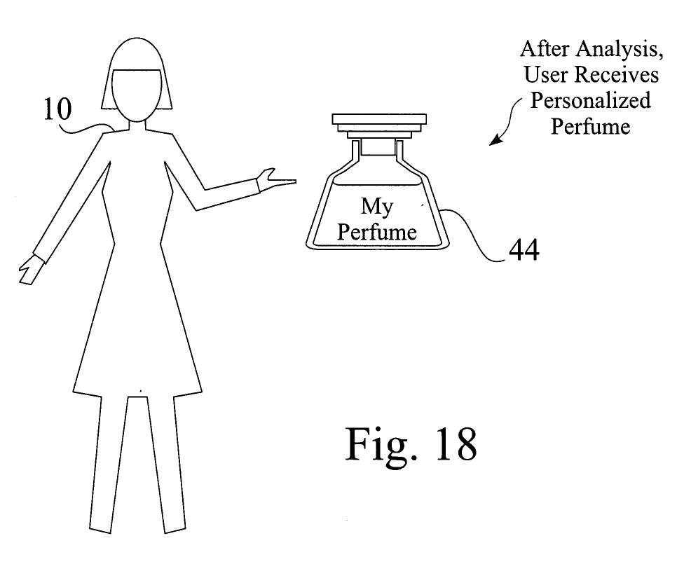

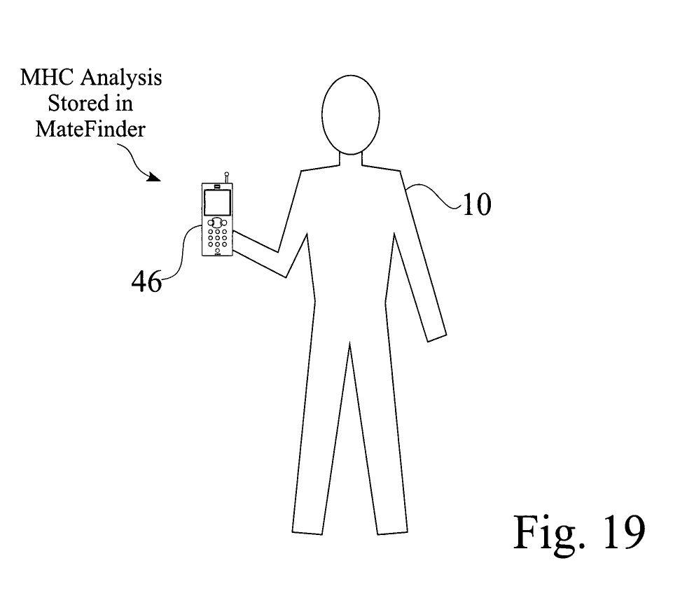

FIG. 19 shows a customer using a MateFinder™ device 46 which has been programmed with his genetic attributes, as determined in accordance with the present invention.

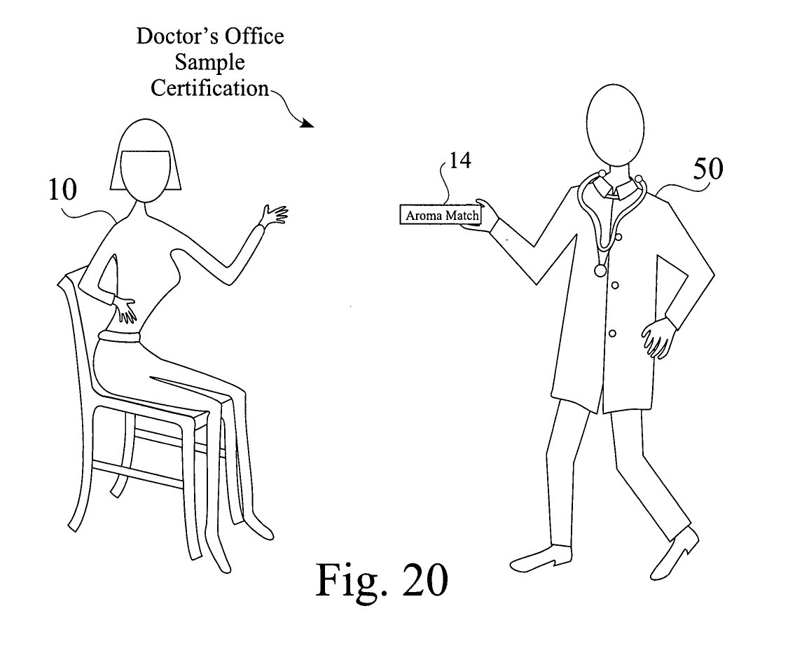

FIG. 20 shows a customer 10 visiting a doctor's office to provide a tissue sample, which is then certified by the doctor 50 before it is submitted to the laboratory. In this embodiment, the physician provides the test kit 14, and obtains the tissue sample. The physician then sends the tissue sample to a laboratory for analysis, and also certifies that the sample is from a particular person. In this example, the physician acts as a “notary” who insures the identity of the source of the sample. This implementation of the invention guards against the fraudulent submission of a tissue sample from a person who might attempt to supply a misleading identity.

II. Custom-Fabricated Perfumes

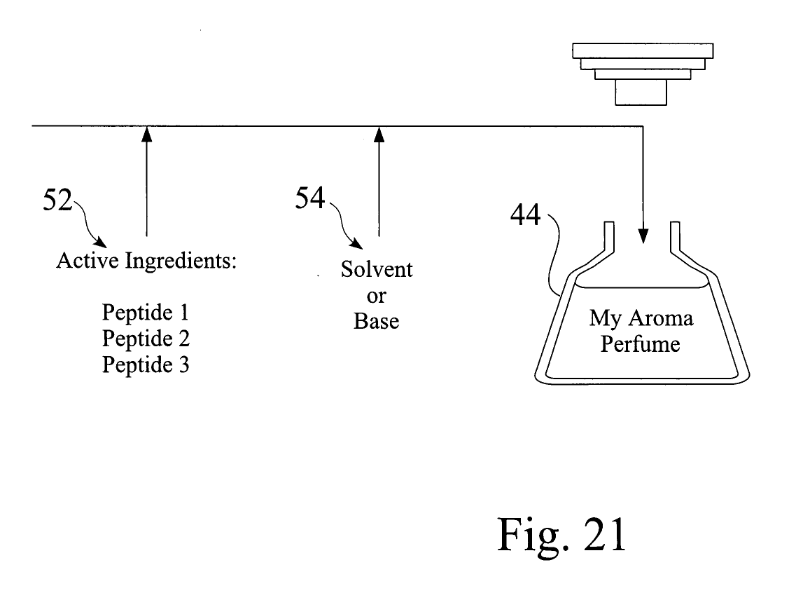

FIG. 21 depicts a method of manufacturing a customized perfume 44. General methods for manufacturing compositions for dispensing fragrances, aromas and perfumes are well known in the art. According to the Scented Products Education and Information Association of Canada, ingredients in a typical fragrance “recipe” generally include:
“extracts from plants and flowers (naturals),
synthetic recreations (synthetic duplications of natural fragrance materials),
synthetic innovations (variations of naturally-occurring materials which have unique olfactory properties).
In general, typical fragrance formulae contain 100-350 ingredients, with an average concentration of usually less than 1%.
In a perfume, ethyl alcohol (of the same grade and purity as in alcoholic beverages) composes 50-90% of the product, purified water may constitute 5-20% of the product, with the fragrance component accounting from 10-30% of the finished product. Also present are UV inhibitors (to prevent degradation in the bottle) and any additional colouring agents.”
   SPEIAC, 20 Britannia Road East, Suite 102, Mississauga, Ontario L4Z 3L5 See: http://www.cctfa.ca/scented/fragmat.htm

In one embodiment of the present invention, appropriate combinations of biological agents such as peptides or other substances are added as active ingredients 52 to a base 54 to a mixture, together with and/or any other suitable solvents, stabilizers, agents, preservatives, dispersants, inhibitors or components. In one embodiment, the base is a solvent, such as alcohol or water. These biological agents are selected to match a genetic attribute possessed by a person.

In one implementation, the perfume or cologne 44 made in accordance with the invention contains substances which are complementary to the user's Major Histocompatibility Complex (MHC profile), which will be attractive to the same user. In the same implementation, that person may ask a spouse or mate to wear this perfume or cologne 44, which pleases the person for whom the customized perfume or cologne was made. The present invention includes both perfume or cologne intended to be used by a person selecting the perfume or cologne for herself or himself, as well as an “inverse perfume or cologne,” which is selected by one person and used by another.

The biological agents may be selected to promote the responsivity of the person using the mixture, or may be selected to promote the responsivity of another person using the mixture. The biological agents in the mixture may be used to broadcast or indicate sexual compatibility, interest, awareness or attraction. As an alternative, the biological agents may be selected to promote confidence, self-esteem or the interest or attraction of another. The invention may be used to promote relationships between members of the opposite sex, or between members of the same sex.

The specific composition of the mixture may take many forms, including, but not limited to a perfume, a cologne, a salve, a paste, an aerosol spray, a powder, or a cosmetic. The cosmetic may include skin cream, lipstick, lip balm, gel, ointment, colorant, or some other preparation that be applied to the body. The mixture is generally intended to be applied to, dispensed on or worn on the skin or hair, but may be applied on or used in conjunction with an article of clothing, which may be impregnated with the active ingredients. In yet another embodiment, the perfume 44 may be encapsulated or contained in a pill or medication that is taken internally, and which is then secreted through the skin or which causes a biological reaction which produces or mimics an odor. The mixture may also be dispensed using a variety of devices, including, but not limited to air fresheners, aroma-dispensing devices, candles and incense.

This specialized perfume 44 contains a strong preparation of personal peptides, enabling the user to “broadcast” his or her “MHC” over a wide area, and increasing his or her chances of meeting a compatible partner. The perfume 44 provides a much more specific set of aromas than, for instance, not bathing for a long period, since odors thus derived have been bacterially modified and contain many non-MHC-related components.

The MHC is a cluster of genes that determines details of cellular surfaces and thus immune responses, and specifies certain peptides that appear in skin secretions and urine. These peptides are responsible for odors which uniquely identify individuals who are not identical twins. Detailed information concerning the MHC may be found in Leslie A. Knapp's publication entitled The ABCs of MHC, published in Evolutionary Anthropology 14:28-37 (2005) Wiley-InterScience. MyAroma™, MyPerfume™, MyEssence™ are Trade & Service Marks owned by the Assignee of the Present Patent Application.

### CONCLUSION

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives for providing a Human Sample Matching System that have been disclosed above are intended to educate the reader about preferred embodiments of the invention, and are not intended to constrain the limits of the invention or the scope of Claims.

### LIST OF REFERENCE CHARACTERS

10 Woman
12 Retail store
14 AromaMatch™ Test Kit
16 Personal computer
18 Website: AromaMatch.com
20 Bottle of cleaning solution
22 Cotton ball
24 Sample patch
24C Central area of patch
24S Strips extending away from central area
25 Plaster
26 Antibiotic
27 Adhesive
28 Sealable plastic bag
30 Mailing envelope
32 Lab technician
34 Sample analyzer
36 Aroma in the air
38 Aroma-sampling kiosk
40 Shopping mall
42 Tissue sample from cheek swab
44 Custom-formulated perfume
46 MateFinder™ device
48 Genetic attributes stored in MateFinder
50 Medical doctor or independent laboratory technician
52 Active ingredient
54 Base

### SEQUENCE LISTING

Not Applicable.

1. A method comprising the steps of:
obtaining a sample (S) from a person (10) using a human sample test kit (14);
analyzing said sample (S) to determine a set of genetic attributes (G1) for said person (10); and
predicting a good match between said person (10) and a member of the opposite sex based on a correlation of said person's set of genetic attributes (G1) and a set of genetic attributes (G2) for a member of the opposite sex.

2. A method as recited in claim 1, in which said sample (S) is obtained from a body tissue.

3. A method as recited in claim 1, in which said sample (S) is obtained from blood.

4. A method as recited in claim 1, in which said sample (S) is obtained from a fingerprint.

5. A method as recited in claim 1, in which said sample (S) is obtained from saliva.

6. A method as recited in claim 1, in which said sample (S) is obtained from urine.

7. A method as recited in claim 1, in which said sample (S) is obtained from hair.

8. A method as recited in claim 1, in which said sample (S) is obtained from a nail.

9. A method as recited in claim 1, in which said sample (S) is obtained from skin.

10. A method as recited in claim 1, in which said human sample test kit (14) includes a skin cleaner (20).

11. A method as recited in claim 1, in which said human sample test kit (14) includes a cleaning medium (22).

12. A method as recited in claim 1, in which said human sample test kit (14) includes a sample patch (24).

13. A method as recited in claim 12, in which said sample patch (24) includes a means for collecting and storing an aroma (25).

14. A method as recited in claim 13, in which said means for collecting and storing an aroma (A) is a portion of plaster (25).

15. A method as recited in claim 12, in which said sample patch (24) includes an adhesive strip (24S).

16. A method as recited in claim 12, in which said sample patch (24) includes an antibiotic (26).

17. A method as recited in claim 12, in which said sample patch (24) is worn on the skin for a period of time that is sufficient to collect and store an aroma (A) that may be reliably analyzed to determine a set of genetic attributes (G1).

18. A method as recited in claim 12, in which said sample patch (24) is worn on the skin for at least eight hours.

19. A method as recited in claim 1, in which said human sample test kit (14) includes a sealable enclosure (28).

20. A method as recited in claim 1, in which said human sample test kit (14) includes a mailing envelope (30).

21. A method as recited in claim 20, in which said mailing envelope (30) is used to return said sample patch (24) to a laboratory for analysis.

22. A method as recited in claim 21, in which a laboratory analysis of said sample patch (24) is conveyed to said person (10) by mail.

23. A method as recited in claim 21, in which a laboratory analysis of said sample patch (24) is conveyed to said person (10) by mail.

24. A method as recited in claim 21, in which a laboratory analysis of said sample patch (24) is conveyed to said person (10) by e-mail.

25. A method as recited in claim 21, in which a laboratory analysis of said sample patch (24) is viewed by said person (10) using a website (16) displayed on a personal computer (18).

26. A method as recited in claim 22, in which a person (10) receives a suggested match based on said laboratory analysis.

27. A method as recited in claim 1, in which said sample (S) is obtained using a cheek swab (SW).

28. A method as recited in claim 1, in which said sample (S) is obtained directly from the air surrounding a person (10).

29. A method as recited in claim 1, in which the identity of said person (10) who provides said sample (S) is authenticated by a third party.

30. A method as recited in claim 1, further comprising the step of:
providing a perfume (44) which has been formulated based on said laboratory analysis of said sample (S).

31. A composition of matter comprising:
an active ingredient (52);
a base (54);
said active ingredient (52) being added to said base (54) to compose a mixture;
said active ingredient (52) including a biological agent;
said biological agent being selected to match a genetic attribute possessed by a person.

32. A composition of matter as recited in claim 31, in which
said base (54) is a solvent.

33. A composition of matter as recited in claim 31, in which
said base (54) is alcohol.

34. A composition of matter as recited in claim 31, in which
said base (54) is water.

35. A composition of matter as recited in claim 31, in which
said biological agent is a peptide.

36. A composition of matter as recited in claim 31, in which
said peptide is selected from a group of peptides related to a cluster of human genes called the Major Histocompatibility Complex.

37. A composition of matter as recited in claim 31, in which
said peptide is selected to promote the responsivity of a person using said mixture.

38. A composition of matter as recited in claim 31, in which
said peptide is selected to promote the responsivity of a person to another person using said mixture.

39. A composition of matter as recited in claim 31, in which
said peptide is selected to promote the confidence of a person using said mixture.

40. A composition of matter as recited in claim 31, in which
said peptide is selected to promote the interest of another person using said mixture.

41. A composition of matter as recited in claim 31, in which
said peptide is selected to promote the attraction of a person using said mixture.

42. A composition of matter as recited in claim 31, in which
said mixture is fabricated as a perfume.

43. A composition of matter as recited in claim 31, in which
said mixture is fabricated as a cologne.

44. A composition of matter as recited in claim 31, in which
said mixture is fabricated as a salve.

45. A composition of matter as recited in claim 31, in which
said mixture is fabricated as an aerosol spray.

46. A composition of matter as recited in claim 31, in which
said mixture is incorporated in a cosmetic.

47. A composition of matter as recited in claim 46, in which said cosmetic is a lipstick.

48. A composition of matter as recited in claim 31, in which said mixture is applied to the skin.

49. A composition of matter as recited in claim 31, in which
said mixture is applied to an article of clothing.

50. A composition of matter as recited in claim 31, in which
said mixture is contained in an air freshener.

51. A composition of matter as recited in claim 31, in which
said mixture is contained in an aroma-dispensing device.

52. A composition of matter as recited in claim 31, in which
said mixture is contained in a candle.

53. A composition of matter as recited in claim 31, in which
said mixture is contained in a piece of incense.

54. A perfume comprising:
a base (54); and
a plurality of active ingredients (52); said plurality of active ingredients (52) being mixed with said base (54);
said plurality of active ingredients (52) being selected base (54) on an analysis of a human sample; said human sample containing information regarding a genetic analysis of a user from whom said sample was obtained;
said plurality of active ingredients (52) being further selected to elicit a sexual response.

55. A perfume as recited in claim 54, which is worn by said user, from whom said human sample was obtained.

56. A perfume as recited in claim 54, which is worn by said user, from whom said human sample was obtained, to broadcast sexual compatibility.

57. A perfume as recited in claim 54, which is worn by another person designated by said user, from whom said human sample was obtained.

58. A perfume as recited in claim 55, which is worn by another person designated by said user, from whom said human sample was obtained, to broadcast sexual compatibility.

59. A perfume as recited in claim 54, which is contained in a medication that is taken internally, and which is then secreted through the skin.

60. A perfume as recited in claim 54, which is contained in a medication that is taken internally, and then which causes a biological reaction which produces an odor.

61. A method as recited in claim 1, further comprising the steps of:
storing said person's set of genetic attributes (G1) and said set of genetic attributes (G2) for a member of the opposite sex in a personal radio device; and
using a personal radio to find a match.
